(19) United States
(12) Patent Application Publication
(10) Pub. No.: US 2010/0269406 A1
(43) Pub. Date: Oct. 28, 2010

(54) Novel Coupled Hydrocarbyl-Substituted Phenol Materials as Oilfield Wax Inhibitors

(75) Inventors: David J. Moreton, Derbyshire (GB); Antonio Mastrangelo, Nottingham (GB); Malcolm MacDuff, Derbyshire (GB)

Correspondence Address: THE LUBRIZOL CORPORATION;ATTN: DOCKET CLERK, PATENT DEPT., 29400 LAKELAND BLVD., WICKLIFFE, OH, 44092, US

(73) Assignee: The Lubrizol Corporation, Wickliffe, OH (US)

(21) Appl. No.: 12/682,603

(22) PCT Filed: Nov. 13, 2008
(86) PCT No.: PCT/US08/83304

§ 371 Date: Jun. 29, 2010

Related U.S. Application Data
(60) Provisional application No. 60/988,445, filed on Nov. 16, 2007.

(51) Int. Cl. C07C 37/20; C10L 1/183

(52) U.S. Cl. 44/450; 568/790; 568/793

(57) ABSTRACT

Paraffin-containing liquid pour point depressants comprising the reaction product of a hydrocarbyl-substituted phenol and an aldehyde wherein: the olefin used in the preparation of the hydrocarbyl-substituted phenol has a high vinylidene content; the reaction between the hydrocarbyl-substituted phenol and the aldehyde is acid or base catalyzed; and/or the reaction further comprises phenol, are particularly useful for treating crude oils which have an initial pour point of 4° C. or higher, decreasing the fluid's pour point and improving the fluid's low temperature handling properties.

### BACKGROUND OF THE INVENTION

The present invention relates to materials useful for lowering the pour point of wax-containing liquid hydrocarbons, and compositions of and methods for preparing the same.

Various types of distillate fuel oils such as diesel fuels, various oils of lubricating viscosity, automatic transmission fluids, hydraulic oil, home heating oils, and crude oils and fractions thereof require the use of pour point depressant additives in order to allow them to flow freely at lower temperatures. Often kerosene is included in such oils as a solvent for the wax, particularly that present in distillate fuel oils. However, demand for kerosene for use in jet fuel has caused the amount of kerosene present in distillate fuel oils to be decreased over the years. This, in turn, has required the addition of wax crystal modifiers to make up for the lack of kerosene. Moreover, the requirement for pour point depressant additives in crude oils can be even more important, since addition of kerosene is not considered to be economically desirable.

Offshore crude oil production often necessitates the flow of crude oil through undersea pipelines. Sub-sea temperatures can be and often are as low as approximately 4° C. These low temperatures in undersea pipelines can cause the heavier paraffinic fraction of the crude oil to become waxy and eventually crystalline. These waxy deposits can constrict the flow of the crude and can block the pipes.

A common way of cleaning the pipes of such crystalline waxy blockages is to send a mechanical “pig” device to bore through the deposits and clear the pipeline. This is a very time consuming and expensive process as oil rig operations must typically be completely shut down in order to complete the cleaning. Such shutdowns are costly due in large part to the lost production.

A chemical additive alternative that can be fed into the pipe lines with the crude oil to maintain flow assurance is desirable. Such additives include wax crystal modifiers such as pour point depressants and wax dispersants which depress the temperature of the formation of wax crystals and can modify the wax morphology, by for example reducing the size of the crystals that form, thus reducing the propensity of the wax to adhere to the pipe-line walls. The cost of the chemical additive treatment of the crude oil is often favourable when offset against potential down-time for pigging and solvent treatment operations.

Currently various materials are being used as pour point depressants (PPD) and wax inhibitors in oilfield applications to address these issues. The materials used normally consist of a polar groups linked to form short hydrocarbyl polymers with long alkyl chains attached to the polar groups, for example alkyl trimers with methylenic bridges and esters of alkyl phenol-aldehyde polymers. Methacyclophanes can also be used. The most commonly found PPD structure is alkylphenol coupled using an aldehyde to give polymers of varying length (as described in Martella et al, EP 0311452B1 and U.S. Pat. No. 5,039,437).

U.S. Pat. No. 5,039,437, Martella et al., Aug. 13, 1991, (and U.S. Pat. No. 5,082,470, Martella et al., Jan. 21, 1992, a division thereof) disclose alkyl phenol-formaldehyde condensates additives for improving the low temperature flow properties of hydrocarbon oils. The polymer composition has a number average molecular weight of at least about 3,000 and a molecular weight distribution of at least about 1.5; in the alkylated phenol reactant the alkyl groups are essentially linear, have between 6 and 50 carbon atoms, and have an average number of carbon atoms between about 12 and 26; and not more than about 10 mole % of the alkyl groups on the alkylated phenol have less than 12 carbon atoms and not more than about 10 mole % of the alkyl groups on the alkylated phenol have more than 2.6 carbon atoms.

U.S. Pat. No. 4,565,460, Dorer, Jr., et al., Jan. 14, 1986, (and U.S. Pat. Nos. 4,559,155, Dec. 17, 1985, 4,565,550, Jan. 21, 1986, 4,575,526, Mar. 11, 1986, and 4,613,342, Sep. 23, 1986, divisions thereof), disclose additive combinations for improving the cold flow properties of hydrocarbon fuel compositions. The composition includes a pour point depressant which can be a hydrocarbyl-substituted phenol of the formula (R*)ₐ—Ar—(OH)_{b} wherein R* is a hydrocarbyl group selected from the group consisting of hydrocarbyl groups of from about 8 to about 39 carbon atoms and polymers of at least 30 carbon atoms. Ar is an aromatic moiety which can include linked polynuclear aromatic moieties represented by the general formula wherein w is an integer of 1 to about 2.0. Each Lng is a bridging linkage of the type including alkylene linkages (e.g., —CH₂— among others).

U.S. Pat. No. 5,171,330, Santoro et al., Dec. 15, 1992, discloses methacyclophanes with substituents, obtained from the condensation reaction of a resorcin with a product containing an aldehyde group, followed by a reaction with halides of organic acids or alkyl halides.

U.S. Pat. No. 5,707,946, Hiebert et al., Jan. 13, 1998, discloses a pour point depressant which is the reaction product of a hydrocarbyl-substituted phenol having a number average of greater than 30 carbon atoms in the hydrocarbyl-substituent, and an aldehyde of 1 to about 12 carbon atoms, or a source therefore.

While such materials as described by the references above can provide wax inhibiting properties to crude oils and other materials, they generally have poor low temperature handling properties over successive cooling cycles. Such cooling cycles simulate the conditions seen by crude oil in the pipelines and represent a continued problem in the oilfield industry. Efforts to improve low temperature handling properties generally result in less effective wax inhibiting performance. The present invention solves this problem by providing novel coupled-alkylphenol materials that give improved low temperature handling properties while maintaining excellent wax inhibiting performance.

### SUMMARY OF THE INVENTION

The invention provides a pour point depressant composition comprising the reaction product of: (a) a hydrocarbyl-substituted phenol which is the reaction product of (i) phenol and (ii) a olefin; and (b) an aldehyde; wherein the olefin has a vinylidene end group content of at least about 10 mole and less than about 85 mole % and wherein the reaction between (a) and (b) is catalyzed with an acid or base catalyst.

The invention further provides the pour point depressant described above where the olefin has a vinylidene end group content of at least about 20 mole % and less than 85 mole % and/or where the olefin is substantially linear.

The invention further provides a liquid fluid composition comprising a wax-containing liquid and about 50 to about 10,000 parts per million by weight of the pour point depressant composition described above.

The invention also provides a pour point depressant composition comprising the reaction product of (a) a hydrocarbyl-substituted phenol which is the reaction product of (i) phenol and (ii) a olefin; and (b) an aldehyde; wherein the reaction between (a) and (b) is catalyzed by a base catalyst.

The invention also provides a pour point depressant composition comprising the reaction product of: (a) a hydrocarbyl-substituted phenol which is the reaction product of (i) phenol and (ii) a olefin; and (b) an aldehyde; and (c) phenol; wherein components (a), (b) and (0 are reacted in any order or simultaneously; wherein the reaction is catalyzed by a base catalyst.

The invention also provides for a method for reducing the pour point of a wax-containing liquid which exhibits an initial pour point of at least 4° C., comprising adding to said liquid a pour-point reducing amount of the one or more of the pour point depressants described above.

### DETAILED DESCRIPTION OF THE INVENTION

The first aspect of the present invention relates to a pour point depressant comprising the reaction product of (a) a hydrocarbyl-substituted phenol, wherein the hydrocarbyl-substituted phenol is the reaction product of (i) phenol and (ii) an olefin, and (b) an aldehyde, wherein the reaction is carried out in the presence of a catalyst. In one embodiment the olefin, which forms the hydrocarbyl group on the phenol, used to prepare (a) the hydrocarbyl-substituted phenol has a vinylidene end group content of at least about 10 mole % and less than about 85 mole %. In another embodiment the reaction between (a) and (b) is carried out in the presence of a base catalyst. In another embodiment, (c) additional phenol is added to the reaction of (a) and (b), where (a), (b) and (c) can be reacted in any order or simultaneously. Further embodiments of the present invention include combinations of these embodiments.

The Hydrocarbyl-Substituted Phenol. Hydrocarbyl-substituted phenols are known materials, as is their method of preparation. When the term “phenol” is used herein, it is to be understood that this term is not generally intended to limit the aromatic group of the phenol to benzene (unless the context so indicates, for instance, in the Examples), although benzene may be a suitable aromatic group. Rather, the term is to be understood in its broader sense to include hydroxyaromatic compounds in general, for example, substituted phenols, hydroxy naphthalenes, and the like. Thus, the aromatic group of a “phenol” can be mononuclear or polynuclear, substituted, and can include other types of aromatic groups as well.

The aromatic group of the hydrocarbyl-substituted phenol compounds can thus be a single aromatic nucleus such as a benzene nucleus, a pyridine nucleus, or a thiophene nucleus, and can also be a polynuclear aromatic moiety. Such polynuclear moieties can be of the fused type; that is, wherein pairs of aromatic nuclei making up the aromatic group share two points, such as found in naphthalene, anthracene, and the azanaphthalenes. Polynuclear aromatic moieties also can be of the linked type wherein at least two nuclei (either mono or polynuclear) are linked through bridging linkages to each other. Such bridging linkages can be chosen from the group consisting of carbon-to-carbon single bonds between aromatic nuclei, ether linkages, keto linkages, sulfide linkages, polysulfide linkages of 2 to 6 sulfur atoms, sulfinyl linkages, sulfonyl linkages, methylene linkages, alkylene linkages, di-(lower alkyl) methylene linkages, lower alkylene ether linkages, alkylene keto linkages, lower alkylene sulfur linkages, lower alkylene polysulfide linkages of 2 to 6 carbon atoms, amino linkages, polyamino linkages and mixtures of such divalent bridging linkages. In certain instances, more than one bridging linkage can be present in the aromatic group between aromatic nuclei. For example, a fluorene nucleus has two benzene nuclei linked by both a methylene linkage and a covalent bond. Such a nucleus may be considered to have 3 nuclei but only two of them are aromatic. Normally, the aromatic group will contain only carbon atoms in the aromatic nuclei per se, although other non-aromatic substitution, such as in particular short chain alkyl substitution, can also be present. Thus methyl, ethyl, propyl, and t-butyl groups, for instance, can be present on the aromatic groups, even though such groups may not be explicitly represented in structures set forth herein.

Specific examples of single ring aromatic moieties include the following:

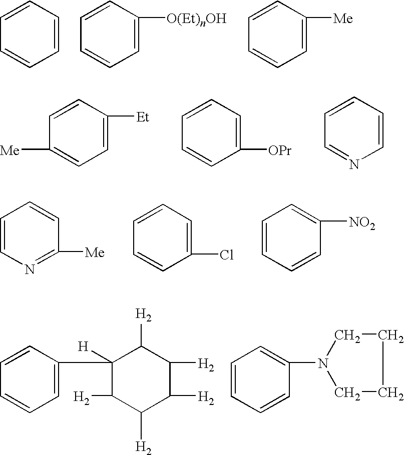

wherein Me is methyl, Et is ethyl or ethylene, as appropriate, and Pr is n-propyl, and wherein the linkage of the aromatic moiety and the hydrocarbyl group that forms the hydrocarbyl-substituted phenol compound may be at, and in place of, any hydrogen atom present on the ring of the moiety.

Specific examples of fused ring aromatic moieties include:

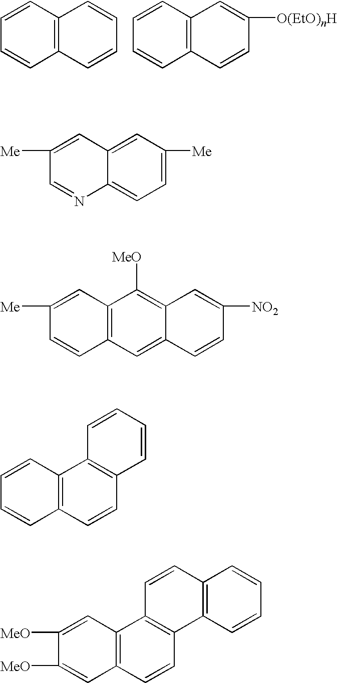

wherein Me is methyl, an Et is ethyl or ethylene, as appropriate; and wherein the linkage of the fused ring aromatic moiety and the hydrocarbyl group that form the hydrocarbyl-substituted phenol compound may be at, and in place of, any hydrogen atom present on the ring of the moiety.

When the aromatic moiety is a linked polynuclear aromatic moiety, it can be represented by the general formula:

ar(-L-ar-)_{w}

wherein w can be an integer of 1 to 20, each ar is a single ring or a fused ring aromatic nucleus of 4 to 12 carbon atoms and each L is independently selected from the group consisting of carbon-to-carbon single bonds between ar nuclei, ether linkages (e.g. —O—), keto linkages (e.g., —C(═O)—), sulfide linkages (e.g., —S—), polysulfide linkages of 2 to 6 sulfur atoms (e.g., —S—₂₋₆, sulfinyl linkages (e.g., —S(O)—), sulfonyl linkages (e.g., —S(O)₂—), lower alkylene linkages (e.g., —CH₂—, —CH₂—CH₂—, —CH₂—CHR^{o}—), mono(lower alkyl)-methylene linkages (e.g., —CHR^{o}—), di(lower alkyl)-methylene linkages (e.g., —CR^{o}₂—), lower alkylene ether linkages e.g., —CH₂O—, —CH₂—O—CH₂—, —CH₂—CH₂—O—, —CH₂CH₂—O—CH₂CH₂—, —CH₂CHR^{o}—O—CH₂CHR^{o}—, —CHR^{o}—O—, —CHR^{o}—O—CHR^{o}—, —CH₂CHR^{o}—O—CHR^{o}—CH₂—), lower alkylene sulfide linkages (e.g., wherein one or more —O—'s in the lower alkylene ether linkages is replaced with a S atom), lower alkylene polysulfide linkages (e.g., wherein one or more —O— is replaced with a —S—₂₋₆ group), amino linkages (e.g., —NH—, —NR^{o}—, —CH₂N—, —CH₂NCH₂—, -alk-N—, where alk is lower alkylene), polyamino linkages (e.g., —N(alkN)1-10, where the unsatisfied free N valences are taken up with H atoms or R^{o} groups), linkages derived from oxo- or keto-carboxylic acids (e.g.)

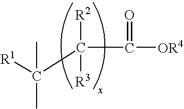

wherein each of R¹, R² and R³ is independently hydrocarbyl, such as alkyl or alkenyl, e.g., lower alkyl, or H; wherein R⁴ is H or an alkyl group and x is an integer ranging from 0 to 8; and mixtures of such bridging linkages (each R^{o} being a lower alkyl group). Unless otherwise defined, the term “lower alkyl” refers to an alkyl group containing 1 to 6 carbon atoms, examples of which are methyl, ethyl, propyl, butyl, amyl and pentyl. In addition to linear alkyl groups, the term includes branched alkyl groups as well, examples of which are isoproyl, isobutyl, sec-butyl, tert-butyl, amyl, isopentyl, and neopentyl.

Specific examples of linked moieties are:

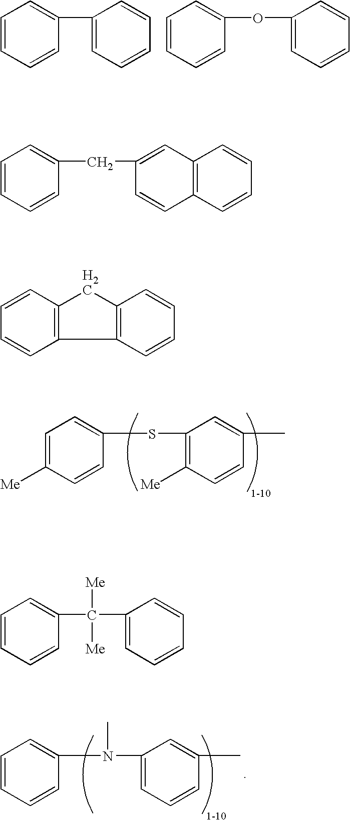

wherein the linkage of the fused ring aromatic moiety and the hydrocarbyl group that form the hydrocarbyl-substituted phenol compound may be at, and in place of, any hydrogen atom present in the ring of the moiety.

Usually all of these aromatic groups have no substituents except for those specifically named. For such reasons as cost, availability, and performance, the aromatic group is normally a benzene nucleus, a lower alkylene bridged benzene nucleus, or a naphthalene nucleus. In certain embodiments the aromatic group is a single benzene nucleus.

This first reactant, the hydrocarbyl-substituted phenol, is a hydroxyaromatic compound, that is, a compound in which at least one hydroxy group is directly attached to an aromatic ring. The number of hydroxy groups per aromatic group will vary from 1 up to the maximum number of such groups that the hydrocarbyl-substituted aromatic moiety can accommodate while still retaining at least one, and typically at least two, positions, at least some of which are typically adjacent (ortho) to a hydroxy group, which are suitable for further reaction by condensation with aldehydes (described in detail below). Thus most of the molecules of the reactant will typically have at least two unsubstituted positions. Suitable materials can include, then, hydrocarbyl-substituted catechols, resorcinols, hydroquinones, and even pyrogallois. Most commonly each aromatic nucleus, however, will bear one hydroxyl group and, in the case when a hydrocarbyl substituted phenol is employed, the material will contain one benzene nucleus and one hydroxyl group. Of course, a small fraction of the aromatic reactant molecules may contain zero hydroxyl substituents. For instance, a minor amount of non-hydroxy materials may be present as an impurity. However, this does not defeat the spirit of the inventions, so long as the starting material is functional and contains, typically, at least one hydroxyl group per molecule.

The hydroxyaromatic reactant is similarly characterized in that it is hydrocarbyl substituted. The term “hydrocarbyl substituent” or “hydrocarbyl group” is used herein in its ordinary sense, which is well-known to those skilled in the art. Specifically, it refers to a group having a carbon atom directly attached to the remainder of the molecule and having predominantly hydrocarbon character. Examples of hydrocarbyl groups include:

(1) hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form an alicyclic radical);

(2) substituted hydrocarbon substituents, that is, substituents containing non-hydrocarbon groups which, in the context of this invention, do not alter the predominantly hydrocarbon substituent (e.g., halo (especially chloro and fluoro), hydroxy, alkoxy, mercapto, alkylmercapto, nitro, nitroso, and sulfoxy);

(3) hetero substituents, that is, substituents which, while having a predominantly hydrocarbon character, in the context of this invention, contain other than carbon in a ring or chain otherwise composed of carbon atoms. Heteroatorus include sulfur, oxygen, nitrogen, and encompass substituents as pyridyl, furyl, thienyl and imidazolyl. In general, no more than two, preferably no more than one, non-hydrocarbon substituent will be present for every ten carbon atoms in the hydrocarbyl group; typically, there will be no non-hydrocarbon substituents in the hydrocarbyl group.

The hydrocarbyl groups of the hydrocarbyl-substituted phenol may be derived from olefins, including long chain olefins, where the olefins are reacted with phenol to form the hydrocarbyl-substituted phenol of the present invention. Suitable long chaion olefins include polyolefins such as polyolefin. A significant portion of the olefins may have vinylidene end groups, where the vinylidene-containing olefin is represented by the following formula:

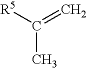

wherein R⁵ is a hydrocarbyl group as described above.

In one embodiment, the olefins used in the invention are substantially linear. In one embodiment the vinylidene end group content of the olefins reacted with phenol to form the hydrocarbyl-substituted phenol is at least about 10 mole % vinylidene end groups, in another embodiment at least about 20 mole % vinylidene groups, and in yet another embodiment at least about 30 mole % vinylidene end groups. In one embodiment, the olefins used to prepare the hydrocarbyl-substituted phenol have a vinylidene end group content of about 45 mole %. In one embodiment, the olefins used are not “high” vinylidene, that is, less than 50 mole %, less than 70 mole %, or less than 85 mole %, of the olefin molecules used contain vinylidene end groups.

The number of carbon atoms present in the olefin and resulting hydrocarbyl group used in the present invention is not particularly limited. In one embodiment the resulting hydrocarbyl group of the hydrocarbyl-substituted phenol will contain 10 to 60 carbon atoms, in one embodiment 12 to 40 carbon atoms, in another embodiment 20 to 40 carbon atoms, and in yet another embodiment 20 to 28 carbon atoms. In one embodiment, the hydrocarbyl group contains on average 12 carbon atoms and in another embodiment the hydrocarbyl group contains 20 to 26 carbon atoms. In some embodiments, the olefins used will be a mixture of olefins, which may vary in length from one particular molecule to another. While a fraction of the molecules may be olefins with a number of carbon atoms outside the ranges described, the composition as a whole will normally be characterized as having less than 30 carbon atoms in length. However, for certain embodiments of the present invention the olefin can be longer, containing up to 400 carbon atoms. In one embodiment the olefin may be larger and contain 31 to 400 carbon atoms, in another embodiment 31 to 60, and in yet another embodiment 32 to 50 or 32 to 45 carbon atoms. The olefin, in any case, may be linear or branched; in one embodiment linear olefins are employed, although the longer chain length materials tend to have increasing proportions of branching.

When using olefins to prepare hydrocarbyl-substituted phenols or alkyl-phenols and thus coupled alkylphenols thereof for use as pour point depressants, a certain amount of branching appears to be introduced due to the migration of the secondary carbocation formed by the catalyst during the substitution or alkylation reaction. The carbocation can migrate down the chain of the olefin. The phenol reacts with the olefins wherever the carbocation is present, thus the migration of the carbocation results in hydrocarbyl-substituted phenols wherein significant amounts of the attached hydrocarbyl groups may be branched.

While not wishing to be bound by theory, it is believed that various embodiments of the present invention, through their use of olefins containing a significant amount of vinylidene end groups in the preparation of hydrocarbyl-substituted phenols and coupled alkylphenols thereof, results in more linear substituent groups on the hydrocarbyl-substituted phenols, as opposed to branched groups. This shift to favoring linear substituent groups is accomplished due to the fact that the vinylidene end group of the olefin used acts to prevent the migration of the carbocation along the olefin chain, keeping the reaction site at or near the end of the olefin. As the reaction site between the phenol and the olefin is kept at or near the end of the olefin, the present invention results in more hydrocarbyl-substituted phenols, and thus more coupled alkylphenols thereof, with linear hydrocarbyl groups attached.

Linear hydrocarbyl groups in the coupled alkylphenol are desirable as it is believed that this feature is preferred in order to permit the chain to more favorably interact with the chain structure of wax-forming hydrocarbons. It is recognized that in many cases there will be one or two methyl branches at the point of attachment of the alkyl chain to the aromatic ring. This is considered to be within the scope of the meaning of a straight chain or linear hydrocarbyl group.

More than one such hydrocarbyl group can be present, but usually no more than 2 or 3 are present for each aromatic nucleus in the aromatic group. Most typically only 1 hydrocarbyl group is present per aromatic moiety, particularly where the hydrocarbyl-substituted phenol is based on a single benzene ring.

The attachment of a hydrocarbyl group to the aromatic moiety of the first reactant of this invention can be accomplished by a number of techniques well known to those skilled in the art. One particularly suitable technique is the Friedel-Crafts reaction, wherein an olefin (e.g., a polymer containing an olefinic bond), or halogenated or hydrohalogenated analog thereof, is reacted with a phenol in the presence of a Lewis acid catalyst. Methods and conditions for carrying out such reactions are well known to those skilled in the art. See, for example, the discussion in the article entitled, “Alkylation of Phenols” in “Kirk-Othmer Encyclopedia of Chemical Technology”, Third Edition, Vol. 2, pages 65-66, Interscience Publishers, a division of John Wiley and Company, N.Y. Other equally appropriate and convenient techniques for attaching the hydrocarbon-based group to the aromatic moiety will occur readily to those skilled in the art.

The Aldehyde. The second component which reacts to form the pour point depressant is an aldehyde of 1 to 12 carbon atoms, or a source thereof. Suitable aldehydes have the general formula RC(O)H, where R may be hydrogen or a hydrocarbyl group, as described above, although R can include other functional groups which do not interfere with the condensation reaction (described below) of the aldehyde with the hydroxyaromatic compound. This aldehyde typically contains 1 to 12 carbon atoms, such as 1 to 4 carbon atoms or 1 or 2 carbon atoms. Such aldehydes include formaldehyde, acetaldehyde, propionaldehyde, butyraldehyde, isobutyraldehyde, pentanal, caproaldehyde, benzaldehyde, and higher aldehydes. Monoaldehydes may be used. A suitable aldehyde is formaldehyde, which can be supplied as a solution, but is more commonly used in the polymeric form, as paraformaldehyde. Polymeric aldehydes, such as paraformaldehyde, may be considered a reactive equivalent of, or a source for, an aldehyde. Aqueous solutions of aldehydes such as formalin may also be considered a reactive equivalent of, or a source for, an aldehyde. Other reactive equivalents may include acetals, hemiacetals, hydrates or cyclic trimers of aldehydes.

The hydrocarbyl-substituted phenol and the aldehyde are generally reacted in relative amounts ranging from molar ratios of hydrocarbyl-substituted phenol:aldehyde of 3:1 to 1:3 and in another embodiment from 2:1 to 1:2. In one embodiment approximately equal molar amounts are employed up to a 30% molar excess of the aldehyde (calculated based on aldehyde monomer), however greater excess aldehyde may be used. In another embodiment the amount of the aldehyde is 5% to 20% or 8% to 15% greater than the substituted phenol on a molar basis. In yet another embodiment the substituted phenol is present at three times the molar amount of the aldehyde or in another embodiment at twice the molar amount of the aldehyde. The components are reacted under conditions to lead to oligomer or polymer formation. The molecular weight of the product will depend on features including the equivalent ratios of the reactants, the temperature and time of the reaction, and the impurities present. The product can have from 2 to 100 aromatic units (i.e., the substituted aromatic phenol monomeric units) present (“repeating”) in its chain, in one embodiment 3 to 70 such units, in another embodiment 4 to 50, 30, or 14 units, and in another embodiment 6 to 8 units.

The molecular weight of the product is not particularly limited and is dependant on the sizes of the olefins used to prepare the substituted phenol and the aldehyde used in the coupling reaction. In one embodiment, where the substituted phenol is prepared from olefins containing 24-28 carbon atoms, and when the aldehyde is formaldehyde, the material will have a number average molecular weight of 1,000 to 24,000. In another embodiment the molecular weight will be 2,000 to 18,000 or in another embodiment 3,000 to 6,000. In yet another embodiment, olefins containing less than 20 carbon atoms may be used to prepare the substituted phenols and formaldehyde can be used as the aldehyde, and the product can have a number average molecular weight of less than 6,000, less than 3000 or less than 2000. In another embodiment the olefins used can contain more than 30 carbon atoms, the aldehyde used can contain up to 12 carbon atoms, and the resulting product can have a number average molecular weight of greater than 6,000, greater than 8,000, or greater than 12,000.

The hydrocarbyl-substituted phenol and the aldehyde are reacted by mixing the components in an appropriate amount of diluent oil or, optionally, another solvent such as an aromatic solvent, e.g., xylene, in the presence of an acid such as sulfuric acid, a sulfonic acid such as an alkylbenzenesulfonic acid, para-toluene sulfonic acid, or methane sulfonic acid, an organic acid such as glyoxylic acid, or Amberlyst™ catalyst, a solid, macroporous, lightly crosslinked sulfonated polystyrene-divinylbenzene resin catalyst from Rohm and Haas. The materials produced by these acid catalyzed reactions can be referred to as Novolaks. The mixture is heated, generally to 75° C. to 160° C., in one embodiment 100° C. to 150° or to 120° C., for a suitable time, such as 30 minutes to 6 hours, or 1 to 4, hours, to remove water of condensation. The time and temperature are correlated so that reaction at a lower temperature will generally require a longer time, and so on. Determining the exact conditions is within the ability of the person skilled in the art. If desired, the reaction mixture can thereafter be heated to a higher temperature, 110° to 180° C., in one embodiment 140° C. to 170° C., and in another embodiment 145° C. to 155° C., to further drive off volatiles and move the reaction to completion. The product can be treated with base such as NaOH if desired, in order to neutralize the strong acid catalyst and to prepare a sodium salt of the product, if desired, and is thereafter isolated by conventional techniques such as filtration, as appropriate. The product of this reaction can be generally regarded as comprising polymers or oligomers having the following structure:

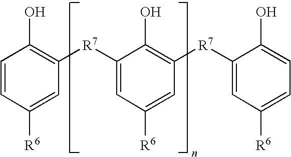

and positional isomers thereof, wherein each R⁶ is independently hydrogen or a hydrocarbyl substituent, each R⁷ is a hydrocarbylene group, and n is, in one embodiment 0 to 98, and in other embodiments 1 to 69, 2 to 49, 2 to 29, 2 to 13 or 4 to 6.

The reaction between the hydrocarbyl-substituted phenol and the aldehyde may also be catalyzed with a base catalyst, such as a metal hydroxide base. Metal hydroxide base suitable for use in the present invention includes, but is not limited to, sodium hydroxide, potassium hydroxide and mixtures thereof. The materials produced by these base catalyzed reactions can be referred to as Resoles and the reaction is carried out a described above. The product of the base catalyzed reaction is the same as the acid catalyzed reaction product except that the base catalyzed reaction product contains a residual methylol group on the last repeating group. The product of this reaction can be generally regarded as comprising polymers or oligomers having the following structure:

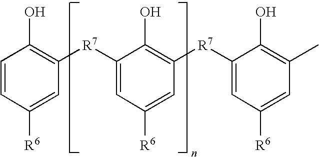

and positional isomers thereof, wherein each R⁶ is independently hydrogen or a hydrocarbyl substituent, each R⁷ is hydrocarbylene group, and n is, in one embodiment 0 to 98, and in other embodiments 1 to 69, 2 to 49, 2 to 29, 2 to 13 or 4 to 6.

The R⁷ linking groups in the two formulas above are derived from the aldehyde used in the coupling reaction. In one embodiment, where the aldehyde used is formaldehyde, the linking group is a methylene group linking the two aromatic groups. In another embodiment, using an aldehyde containing 1 to 12 carbon atoms, the derived linking group also contains 1 to 12 carbon atoms.

Also contemplated in the invention are embodiments where the R⁶ groups in the formulas above are mixtures of linear and branched alkyl groups. These alkyl groups include polyisobutylene groups and polyisobutylene groups with high vinylidene contents. In another embodiment, the R⁶ groups may also contain phenols

However, in both the acid and base catalyzed reactions, when formaldehyde is employed, a portion of the formaldehyde is believed to be incorporated into the molecular structure in the form of substituent groups and linking groups such as those illustrated by the following types, including ether linkages and hydroxymethyl groups:

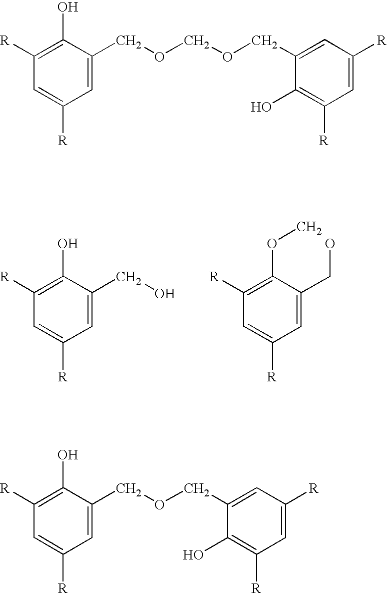

wherein these various linkages between the hydrocarbyl phenols are present with the linkages shown in the general structures above and each R is a hydrocarbyl group. Similar alternative linking is also possible when aldehydes other than formaldehyde are used. The number of carbon atoms present in the alternative linkages shown above depending on the number of carbon atoms in the aldehyde used.

The amount of catalyst used in the preparation of the present invention is not particularly limited, whether the reaction is acid catalyzed or base catalyzed, and can be 1,000 to 50,000 ppm by weight of the reactants present. In one embodiment the catalyst is present from 5,000 to 20,000 ppm by weight of the reactants present, and in another embodiment from 7,000 to 19,000 ppm. In one embodiment, when the catalyst used is a base catalyst, the catalyst may be present at 10,000 to 30,000 ppm, and in another embodiment from 15,000 to 20,000 ppm by weight of the reactants. In another embodiment, when the catalyst used is an acid catalyst, the catalyst may be present at 5,000 to 20,000 ppm, and in another embodiment from 7,000 to 13,000 ppm by weight of the reactants.

The reaction of the hydrocarbyl-substituted phenol and the aldehyde may also be carried out with an amount of non-substituted phenol present. This phenol is separate from the phenol used in the preparation of the hydrocarbyl-substituted phenol. The addition of phenol may be employed whether the coupling reaction is acid or based catalyzed. While not wishing to be bound by theory, it is thought that adding phenol, ortho-substituted phenol, or mixtures thereof, to the reaction of the hydrocarbyl-substituted phenol and the aldehyde results in a tri-substituted product. A proposed structure of one such product is shown in the formula below:

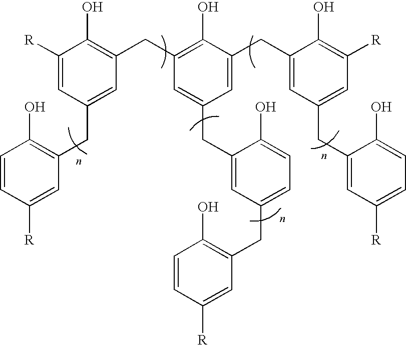

wherein each R is a hydrocarbyl group and n is a number from 0 to about 8. The amount of additional phenol added to the reaction is not particularly limited. However, in one embodiment phenol is added such that it makes up about 0.1 to about 10% by weight of the reactants, and in another embodiment from about 1 to about 5% by weight of the reactants, and in yet another embodiment from about 1.5% to about 3% by weight of the reactants.

Preparation of the pour point depressants by the above method provides a material which generally exhibits improved low temperature handling properties, as evaluated by thermal cycling tests, compared with pour point depressants prepared by prior art methods.

The pour point depressant materials of this invention are particularly suitable for reducing the pour point of certain petroleum oils, i.e., crude oils or fractions of crude oil, such as residual oil, vacuum gas oil, or vacuum residual oils (Bunker C crude oils), that is, naturally sourced and partially refined oils, including partially processed petroleum derived oils. The suitable oils are generally those which have an initial (that is, unmodified, or prior to treatment with the pour point depressant) pour point of at least 4° C. (40′F), preferably at least 10° C. (50° F.) or more preferably 16° C. (60° F.), although they also exhibit some advantage in certain oils which fall outside of these limits. The use of the present materials is particularly valuable in those crude oils which are difficult to treat by other means. For example, they are particularly useful in oils (crude oils and oil fractions such as those described above) which have a wax content of greater than 5%, such as greater than 10%, by weight as measured by UOP-46-85 (procedure from UOP, Inc., “Paraffin wax content of petroleum oils and asphalts”). (Wax-containing materials are sometimes also referred to as paraffin-containing materials, paraffin being an approximate equivalent for wax, and in particular, for petroleum waxes. The present invention is not particularly limited to any specific type of wax which may cause the pour point phenomenon in a given liquid. Thus paraffin wax, microcrystalline waxes, and other waxes are encompassed. It is recognized that in many important materials, such as petroleum oils, paraffin wax may be particularly important.) The pour point depressant materials are further useful in oils with a large high-boiling fraction, that is, in which the fraction boiling between 271° C. (520° F.) and 538° C. (1000° F.) (i.e., about C₁₅ and above) comprises at least 25%, or at least 30%, or at least 35% of the oil (exclusive of any fraction of 7 or fewer carbon atoms). Among high boiling oils, they are more particularly useful if greater than 10%, or greater than 20%, or greater than 30%, of the high boiling (271-538° C.) fraction boils between 399° C. (750° F.) and 538° C. (1000° F.) (i.e., about C₂₋₅ and above), as measured by ASTM D 5307-92. In certain embodiments, this highest boiling (399-538° C.) fraction will comprise at least 10% of the total oil (exclusive of any fraction of 7 or fewer carbon atoms). The analysis may be performed on stock tank crude which is degassed and contains little or no fraction of C₄ or below. They are further useful in materials which have an API gravity of greater than 20° (ASTM D-287-82).

The present pour point depressant material are, in many cases, useful for treating oils (e.g., crude oils and fractions thereof) which have a N_{w} of greater than 18, preferably greater than 20, and more preferably greater than 22. Here N_{w} is the weight average number of carbon atoms of the molecules of the oil, defined by

$${N}_{w} = \frac{\sum {B}_{n} * {n}^{2}}{\sum {B}_{n} * n}$$

where Bₙ represents the weight percent of the crude boiling fraction of the oil containing the alkane CₙH₂ₙ₊₂ and n is the carbon number of the corresponding paraffin. These boiling fraction values are determined by ASTM procedure D5307-92. In certain embodiments, the suitable oils will have the above defined value of N_{w}, as well as one or more of the above-defined characteristics such as a pour point above 4° C. and/or a wax content of greater than 5% (UOP-41-85 procedure).

The amount of the pour point depressant employed in the oil or in the other wax-containing liquid, will be an amount suitable to reduce the pour point thereof by a measurable amount, i.e., by at least 0.6° C. (1° F.), such as at least 2° C. (3 or 4° F.) or 3° C. (5° F.), or even 6° C. (10° F.). This reduction in pour point can be readily determined by one skilled in the art by employing the methodology of ASTM D-97. Typically the amount of pour point employed will be 50 to 10,000 parts per million by weight (ppm), preferably 100 to 5000 ppm, more preferably 200 to 2000 ppm, based on the fluid to which it is added.

The pour point depressants of the present invention can be supplied in the pure form (containing 0% diluent) or as concentrates containing a diluent such as a hydrocarbon oil or solvent. When supplied as a concentrate, the amount of oil can be up to 90% by weight of the composition, typically 10-90%, such as 30-70% or 40-60%, all by weight. In one embodiment the pour point depressant is diluted to provide a blend with a 15% actives content by weight. Alternatively, the pour point depressants can be supplied as dispersions in such materials as acetates (e.g., as 2-ethoxyethyl acetate) or aqueous glycol mixtures (e.g., mixtures of ethylene glycol and water).

### EXAMPLES

### Example 1

A coupled alkylphenol derived from a C20-26 linear alpha olefin with 45% vinylidene content, (this olefin is available from Ineos™), 50% actives in SN40 lube oil.

Step 1—An alkylphenol is prepared by charging to a 3 L flask under nitrogen: 236 grams of toluene, 1052 grams (11.2 moles) of phenol, and 113 grams of an acid treated clay catalyst. The flask is heated to 70° C. and stirred until the components are fully melted and well mixed. The flask is then heated 150° C. over about 50 minutes and is then held for 3 hours. The flask is then heated to 155° C. and 1200 grams (3.73 moles) of 45% vinylidene C20-26 linear alpha olefin is added to the flask over about 65 minutes. The flask is then heated to 165° C. and held under reflux for eight hours. The reaction mixture is then cooled and filtered to give a clear golden/brown liquid. The filtered material is poured into a 3 L flask and heated to 85° C. over about 15 minutes and then held under vacuum for 30 minutes to strip any remaining solvent. The flask is then heated to 200° C. over about 90 minutes for then held for 2 hours, while still under vacuum, to remove any excess phenol. The flask is then cooled and the alkylphenol, a clear brown liquid, is discharged.

Step 2—A coupled alkylphenol is prepared in a 3 L flask under nitrogen by charging 1000 grams (2.4 moles) of the C20-26 alkylphenol prepared above and heating to 85° C. To the flask, 9.0$ grams of sulphuric acid is added drop-wise over about 2 minutes, and the reaction mixture is held at 85° C. for 30 minutes. The flask is then heated to 105° C. and 74.26 grains (2.475 moles) paraformaldehyde is charged over 45 minutes. The flask is then heated to 120° C. and held for 2 hours to collect any aqueous distillate. Over 90 seconds, 14.92 grams of a 50% by weight aqueous solution of sodium hydroxide is added dropwise. The flask is then heated to 150° C. and held for a further two hours to allow the reaction to complete and collect any additional and/or remaining aqueous distillate. The flask is then cooled to about 90° C. and 1072 grams of petroleum naphtha solvent is charged to the flask. After 30 minutes the mixture is discharged and filtered to give a dark oil with a kinematic viscosity at 100° C. of 8.6 mm²/s (cSt), a pour point of −39° C. and a number average molecular weight of 6,780 determined by GPC.

### Example 2

A coupled alkylphenol derived from a C12 alkylphenol derived from a linear alpha olefin with less than 10% vinylidene content.

Step 1—An alkylphenol is prepared by the steps described above in Example 1, Step 1, using 794.8 grams (4.72 moles) 1-dodecene, 1333.3 grams (14.16 moles) phenol, 145.1 grams acid treated clay catalyst, and 1396.7 grams toluene.

Step 2—A coupled alkylphenol is prepared by the steps described above in Example 1, Step 2, using 550 grams (2.096 moles) alkylphenol from Example 2, Step 1, 64.83 grams (1.03 moles) paraformaldehyde, 7.91 grams sulphuric acid, 13.01 grams of a 50% by weight aqueous solution of sodium hydroxide and 590 grams of petroleum naphtha solvent. The only difference in the steps described in Example 1, step 2 is that the solvent is added at the start of the reaction with the alkylphenol rather than at the end of the reaction. The mixture is collected and to give a dark oil with a Kinematic viscosity at 100° C. of 6.7 mm²/s (cSt), a pour point of 51° C. and a number average molecular weight of 3,086 determined by GPC.

### Example 3

A coupled alkylphenol derived from a C24-28 alkyphenol.

A coupled alkylphenol is prepared by charging to a 21, flask under nitrogen, 750 grains (1.61 moles) of C24-28 alkylphenol, 18.1 grams of aqueous potassium hydroxide and 790.9 grams of petroleum naphtha solvent. The flask is then heated to 65° C. Then 215.5 grams (2.66 moles) of formalin is added over about one hour. The flask is then heated to 75° C. and held for two hours. The flask is then mixed slowly while 500 grams of water is added and then held for about 5 minutes. The flask is then allowed to settle and the resulting layer of water at the bottom of the flask is removed. The mixture is heated slowly to 110° C. and then to 140° C., where it is held for 2 hours. Vacuum is then applied to remove any remaining aqueous distillate. Once no additional aqueous distillate is being removed from the flask, vacuum is released and the flask is cooled. The mixture is discharged and filtered to give a dark oil with a Kinematic viscosity at 100° C. of 4.1 mm²/'s (cSt), a pour point of 24° C. and a number average molecular weight of 3,677 determined by GPC.

### Example 4

A tri-substituted coupled alkylphenol derived from a C24-28 alkyphenol.

A coupled alkylphenol is prepared by the steps described above in Example 1, Step 2, using 871.1 grams (1.873 moles) C24-28 alkylphenol, 68.6 grams (2.289 moles) paraformaldehyde, 7.07 grams sulphuric acid, 11.62 grams of a 50% by weight aqueous solution of sodium hydroxide and 1088 grams of petroleum naphtha solvent. In addition, 19.6 grams (0.207 moles) of phenol is added to the flask at the same time as the C24-28 alkylphenol. The mixture is discharged and filtered to give a dark oil with a Kinematic viscosity at 100° C. of 9.7 mm²/s (cSt), a pour point of 18° C. and a number average molecular weight of 7,645 determined by GPC.

Two laboratory tests are used to evaluate the low temperature handling properties of the materials of the present invention. These tests are selected to mimic the conditions encountered in the field, where the samples must maintain their low temperature characteristics over successive cooling cycles.

A comparative sample of a commercially available pour point depressant, comprising coupled alkylphenol is included in the testing. The comparative sample contains a coupled alkylphenol derived from a C24-28 linear alpha olefin, using an acid catalyst. The comparative sample is not derived from hydrocarbyls with vinylidene end groups, is not the result of a base catalyzed coupling reaction, and no additional phenol is present during the coupling reaction of the product. Generally, the comparative sample material is used in the field in a diluted form where a solvent or other diluent is added to give a blend with 15% actives material. The examples described above are all diluted to this actives level for the testing by adding an appropriate amount of petroleum naphtha solvent.

The examples are evaluated in a triple pour point test. The pour point testing is carried out on an ISL CPP 97-6 Automatic Pour Point Apparatus to method ASTM D5950, which is an automated version of ASTM D97. After preliminary heating, the test sample, in a jar, is inserted into the automatic pour point apparatus. The sample is then cooled and examined at 3° C. intervals; the instrument tilts the test jar and detects movement of the surface of the sample with an optical device. The lowest temperature at which movement of sample is detected is recorded as the pour point.

To determine low temperature characteristics during successive cooling cycles, the pour point of the examples above are measured in triplicate, the sample being heated back to room temperature between each pour point measurement. Materials with good low temperature handling properties across successive cooling cycles would be expected to have pour points at least as low as the comparative example and to be able to maintain their low pour point for all three measurements. Table 1 below shows the triple pour point data for the examples along with the comparative example.

TABLE 1
Triple Pour Point Test Results

| | Pour | Pour | Pour |
|---|---|---|---|
| | Point 1 | Point 2 | Point 3 |
| Example | ° C. | ° C. | ° C. |
| Comparative | −3 | 0 | −3 |
| 1 | <−54 | −54 | −54 |
| 2 | −51 | −57 | −54 |
| 3 | −24 | −27 | −24 |
| 4 | −9 | −9 | −9 |

All of the examples show lower pour points than the comparative example. These results indicate that the present invention provides improved low temperature handling properties and maintains those properties over successive cooling cycles while still providing good wax inhibition.

The second test carried out is a thermal cycle cooling test. Here, all samples are again diluted to a 15% actives level using petroleum naphtha solvent. The test samples, in jars, are stored in an oven at 50° C. to normalize, with regards to dissolution of any crystalline material already formed. The jars are then stored in a freezer at −15° C. for 20 hours, after which the sample are removed from the freezer and rated, where S is for solid, L is for liquid, and G is for gelatinous. The jars are then held at room temperature (RT) for 2 hours and then rated again. The samples are the returned to the freezer at −15° C. This process is repeated until three sets of ratings are collected. Good performers would be expected to remain liquid throughout the testing. Table 2 below shows the thermal cycling data for the examples along with the comparative example.

TABLE 2
Thermal Cycling test results

| | Appearance of test sample during thermal cycling test | | | | | |
|---|---|---|---|---|---|---|
| | Cycle 1 | | Cycle 2 | | Cycle 3 | |
| Example | at −15 C. | at RT | at −15 C. | at RT | at −15 C. | at RT |
| Comparative | S | L | S | L | S | L |
| 1 | L | L | L | L | L | L |
| 2 | L | L | L | L | L | L |
| 3 | S | L | S | L | S | L |
| 4 | S | L | S | L | S | L |

Examples 1 and 2 perform better than the comparative sample in the thermal cycling test, remaining liquid at −15° C. throughout the cycles. Examples 3 and 4 perform as well as the comparative sample, as they are solid at −15° C. after each cycle. These results indicate that the present invention provides improved low temperature handling properties and maintains those properties over successive cooling cycles while still providing good wax inhibition.

Each of the documents referred to above is incorporated herein by reference. Except in the Examples, or where otherwise explicitly indicated, all numerical quantities in this description specifying amounts of materials, reaction conditions, molecular weights, number of carbon atoms, and the like, are to be understood as modified by the word “about.” Unless otherwise indicated, each chemical or composition referred to herein should be interpreted as being a commercial grade material which may contain the isomers, by-products, derivatives, and other such materials which are normally understood to be present in the commercial grade. However, the amount of each chemical component is presented exclusive of any solvent or diluent oil which may be customarily present in the commercial material, unless otherwise indicated. As used herein, the expression “consisting essentially of” permits the inclusion of substances which do not materially affect the basic and novel characteristics of the composition under consideration.

What is claimed is:

1. A pour point depressant composition comprising the reaction product of:
(a) a hydrocarbyl-substituted phenol which is the reaction product of (i) phenol and (ii) a olefin; and
(b) an aldehyde;
wherein the olefin has a vinylidene end group content of at least about 10 mole % and less than about 85 mole %.
wherein the reaction between (a) and (b) is catalyzed, and wherein the catalyst comprises an acid catalyst or a base catalyst.

2. The composition of claim 1 wherein the composition is further reacted with (c) phenol; wherein components (a), (b) and (c) are reacted in any order or simultaneously.

3. The composition of claim 1 wherein (a)(ii), the olefin, has a vinylidene end group content of at least about 20 mole % and less than 85 mole %.

4. The composition of claim 1 wherein the catalyst is an acid catalyst which comprises: sulfuric acid; a sulfonic acid, a carboxylic acid, or combinations thereof.

5. The composition of claim 1 wherein the catalyst is a base catalyst which comprises metal hydroxide base and mixtures thereof.

6. The composition of claim 1 wherein (b), the aldehyde, is formaldehyde, paraformaldehyde, acetaldehyde, propionaldehyde, butyraldehyde, isobutyraldehyde, pentanal, caproaldehyde, benzaldehyde, a source thereof, or mixtures thereof.

7. The composition of claim 1 wherein the reaction product comprises the reaction of the hydrocarbyl-substituted phenol and the aldehyde or source thereof in a molar ratio of about 2:1 to about 1:1.5.

8. The composition of claim 3 wherein the olefin comprises a mixture of molecules having predominantly 12 to 2.6 carbon atoms.

9. A liquid fluid composition comprising a wax-containing liquid and about 50 to about 10,000 parts per million by weight of the pour point depressant composition of claim 1.

10. A pour point depressant composition comprising the reaction product of:
(a) a hydrocarbyl-substituted phenol which is the reaction product of (i) phenol and (ii) a olefin; and
(b) an aldehyde;
wherein the reaction between (a) and (b) is catalyzed by a base catalyst.

11. A pour point depressant composition comprising the reaction product of:
(a) a hydrocarbyl-substituted phenol which is the reaction product of (i) phenol and (ii) a olefin; and
(b) an aldehyde; and
(c) phenol;
wherein components (a), (b) and (c) are reacted in any order or simultaneously; wherein the reaction is catalyzed by a base catalyst.

12. A method for reducing the pour point of a wax-containing liquid which exhibits an initial pour point of at least 4° C., comprising adding to said liquid a pour-point reducing amount of a pour point depressant comprising the reaction product of:
(a) a hydrocarbyl-substituted phenol, wherein the hydrocarbyl-substituted phenol is the reaction product of (i) phenol and (ii) a olefin, which has a vinylidene end group content of at least about 10 mole % and less than about 85 mole %; and
(b) an aldehyde;
wherein the reaction between (a) and (b) is acid catalyzed or base catalyzed.

13. The method of claim 12 wherein the reaction producing the pour point depressant further comprises (c) phenol, wherein components (a), (b) and (c) are reacted in any order or simultaneously.

14. A method for reducing the pour point of a wax-containing liquid which exhibits an initial pour point of at least 4° C., comprising adding to said liquid a pour-point reducing amount of a pour point depressant comprising the reaction product of:
(a) a hydrocarbyl-substituted phenol, which is the reaction product of (i) phenol and (ii) a olefin; and
(b) an aldehyde;
wherein the reaction between (a) and (b) is base catalyzed.

15. A method for reducing the pour point of a wax-containing liquid which exhibits an initial pour point of at least 4° C., comprising adding to said liquid a pour-point reducing amount of a pour point depressant comprising the reaction product of:
(a) a hydrocarbyl-substituted phenol, which is the reaction product of (i) phenol and (ii) a olefin; and
(b) an aldehyde; and
(c) phenol;
wherein components (a), (b) and (c) are reacted in any order or simultaneously; wherein the reaction is acid catalyzed or base catalyzed.
